(19) United States
(12) Patent Application Publication
(10) Pub. No.: US 2020/0321105 A1
(43) Pub. Date: Oct. 8, 2020

(54) SYSTEM AND METHODOLOGIES FOR IMPROVED PATIENT TREATMENT MANAGEMENT AND PROCESSING

(71) Applicant: PCGI Systems, Potomac, MD (US)

(72) Inventor: Abhijit PARAB, Potomac, MD (US)

(73) Assignee: Parab Consulting Group Inc. DBA PCGI, Potomac, MD (US)

(21) Appl. No.: 16/829,506

(22) Filed: Mar. 25, 2020

Related U.S. Application Data
(60) Provisional application No. 62/823,234, filed on Mar. 25, 2019.

(51) Int. Cl. G16H 40/20; G06Q 40/08; G06F 3/0483

(52) U.S. Cl. CPC G16H 40/20; G06F 3/0483; G06Q 40/08

(57) ABSTRACT

A system and various methodologies provide the ability to perform various patient related functionality implemented in a cloud computing environment, for example, patient onboarding, case management, task management and next best action evaluation of health benefits and prior authorization, treatment planning, patient adherence metric analysis and other associated reporting and analytics functionality.

### CROSS-REFERENCE TO RELATED APPLICATIONS

The present application claims priority to U.S. Provisional Patent Application No. 62/823,234, entitled “SYSTEM AND METHODOLOGIES FOR IMPROVED PATIENT TREATMENT MANAGEMENT AND PROCESSING, filed Mar. 25, 2019, incorporated herein by reference in its entirety.

### FIELD

The present disclosure relates to systems, components, and methodologies for analyzing trends and formulating analytics based on customer and sales data across a plurality of data sets.

### BACKGROUND

Challenges posed by pharmaceutical and therapeutics pricing pressure, federal regulations, third party payer (e.g., insurance) reimbursement delays, increased cost of care and a significant increase in expensive rare disease treatments have created significant confusion and frustration in the market place for patient/insureds (i.e., those people seeking and/or receiving medical evaluation and/or treatment). As a result of the complex and disparate technical systems and protocols used by public and private care providers, pharmaceutical and other medical product and service providers, and the insurance industry, there appears to be no way to improve the ability to effectively provide patient/insureds (hereafter collectively referred to as “patients”) with appropriate treatment in an appropriate time period.

Indeed, the Affordable Care Act and other governmental solutions, meant to eliminate such problems, appear to merely reduce the burden on the poorest population of patients but do not truly address the underlying issue, which appears to be how to get patients on the right treatment at the right time to reduce illness and injury in a cost effective manner.

### SUMMARY

As a result, disclosed embodiments provide functionality to perform various customer, i.e., patient related functionality implemented in a cloud computing environment, for example, patient onboarding, case management, task management, artificial intelligence and next best action evaluation of health benefits and prior authorization, treatment planning, patient adherence metric analysis and other associated reporting and analytics functionality.

In accordance with various disclosed embodiments, this functionality may be implemented with a front end that includes a user interface that includes various web and mobile device based screens providing input and/or output functionality to a user for inputting and/or reviewing output data regarding one or more of the above-identified patient, insured related functionality.

Additional features of the present disclosure will become apparent to those skilled in the art upon consideration of illustrative embodiments exemplifying the best mode of carrying out the disclosure as presently perceived.

### BRIEF DESCRIPTION OF THE FIGURES

The detailed description particularly refers to the accompanying figures in which:

FIG. 1 illustrates an example of patient onboarding processes wherein a referral processing utility leverages real time integration with an array of incoming referral methods in accordance with disclosed embodiments.

FIG. 2 provides an illustrative example of the technical utility provided by the disclosed embodiments and the underlying platform so as to highlight the technical problems to which the present innovation provides a technical solution.

FIG. 3 illustrates details of disclosed embodiments that provide a cloud-implemented system and functionality that enables task management, workflow management and next best action that enable offering a robust task management approach to aid in the patient journey.

FIG. 4 illustrates an example of cloud-implemented system functionality for enabling a solution that drives a comprehensive workflow for benefits investigators and treatment initiation teams to enable integration with industry leaders to obtain policy and coverage details for a patient.

FIG. 5 illustrates an example of the reporting, analytics and data services that may be provided by the disclosed embodiments.

FIG. 6 provides an illustrative example of the architecture of the cloud-based system.

FIG. 7 illustrates an example a fax processing dashboard provided in accordance with the disclosed embodiments.

FIG. 8 illustrates an example of a fax processing utility user interface in accordance with disclosed embodiments.

FIGS. 9-12 illustrate examples of patient view user interface screens included in a patient dashboard configured to enable input of various data associated with a patient and review of such and related information accessed via that input in accordance with disclosed embodiments.

FIGS. 13-14 illustrate examples of case view user interface screens included in a case view dashboard configured to enable input of various data associated with a particular case and review of such and related information accessed via that input in accordance with disclosed embodiments.

FIG. 15 illustrates an example of an audit trail on case dashboard configured to enable input of various data associated with a patient's case and review of such and related information accessed via that input in accordance with disclosed embodiments.

FIG. 16 illustrates an example of a Health Care Professional (or Provider (HCP) Information on Case dashboard configured to enable input of various data associated with health care provider(s) on a particular case and review of such and related information accessed via that input in accordance with disclosed embodiments.

FIGS. 17-28 illustrate examples of various user interfaces configured to enable input of various data associated with individual components of a patient's case and review of such and related information accessed via that input in accordance with disclosed embodiments.

### DETAILED DESCRIPTION

The figures and descriptions provided herein may have been simplified to illustrate aspects that are relevant for a clear understanding of the herein described systems, and methods, while eliminating, for the purpose of clarity, other aspects that may be found in typical devices, systems, and methods. Those of ordinary skill may recognize that other elements and/or operations may be desirable and/or necessary to implement the devices, systems, and methods described herein. Because such elements and operations are well known in the art, and because they do not facilitate a better understanding of the present disclosure, a discussion of such elements and operations may not be provided herein. However, the present disclosure is deemed to inherently include all such elements, variations, and modifications to the described aspects that would be known to those of ordinary skill in the art.

Providing a mechanism for putting patients on the right treatment at the right time to reduce illness and injury in a cost effective manner requires collaboration among pharmaceutical and therapeutic manufacturer (which includes manufacturers of pharmaceuticals, other therapeutics and other products used in the medical evaluation or treatment of a patient's health, physical condition, injury, state of disease or other condition pertaining to health; often short-handed as pharmaceutical and therapeutic manufacturers and hereafter referred to as “P&T manufacturers”), the medical provider, insurance provider, pharmacies and the ultimate patient.

Conventionally, the obstacles to providing such seamless collaboration are numerous and complex. As a result, conventionally, there has been no way to provide a technological solution to enable such collaboration. To the contrary, disclosed embodiments provide a system, constituent components and methodologies to enable provisioning of patient services in a cloud environment wherein an Artificial Intelligence (AI) driven hub connects data from a P&T prescriber, patient, care giver, payor, pharmacy and P&T manufacturer, and enables a patient care journey that enables navigation through intelligent and timely decision making.

As a result, disclosed embodiments provide a technical solution to the real world problem in the medical care industry wherein, there is no real time data exchange between patient service hubs, call centers, specialty pharmacies, third party payers and P&T manufacturers.

Moreover, disclosed embodiments provide a technical solution to the real world problem in the medical care industry wherein, there is no ability to provide timely updates to patients regarding their treatment and reimbursement options.

Additionally, disclosed embodiments provide a technical solution to the real world problem in the medical care industry wherein, there is no meaningful and reliable way to determine or predict a delay in the process and propose of a next best action or alternate route to treatment.

Furthermore, disclosed embodiments provide a technical solution to the real world problem in the medical care industry that there is no mechanism for P&T sales representatives, reimbursement managers and third party payer account directors to work in tandem with health care provider and patient to address and overcome prior authorization hurdles.

Finally, disclosed embodiments provide a technical solution to the real world problem in the medical care industry wherein, there is no mechanism for digital collaboration with the patient such that any interaction and involvement of the patient in their care is limited and results in the current process not providing “patient centric” analysis, which could greatly improve the potential for patient adherence to P&T or other medical therapy.

Disclosed embodiments provide functionality to perform various customer, i.e., patient related functionality implemented in a cloud computing environment, for example, patient onboarding, case management, task management and next best action evaluation of health benefits and prior authorization, treatment planning, patient adherence metric analysis and other associated reporting and analytics functionality.

In accordance with various disclosed embodiments, this functionality may be implemented with a front end that includes a user interface that includes various web and mobile device based screens providing input and/or output functionality to a user for inputting and/or reviewing output data regarding one or more of the above-identified patient, insured related functionality.

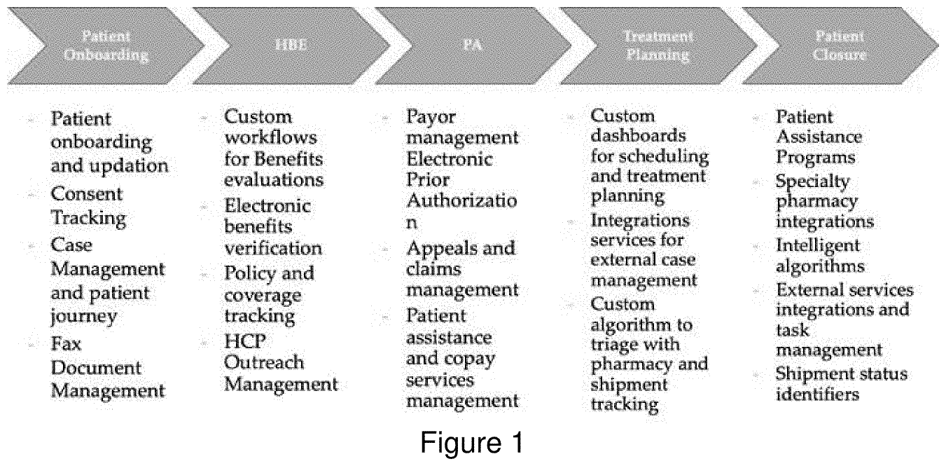

FIG. 1 illustrates an example of patient onboarding processes wherein a referral processing utility leverages real time integration with an array of incoming referral methods (e.g., fax, web portals and other electronic services), and wherein a referral, i.e., a patient, is referred into a system provided in accordance with the disclosed embodiments. Accordingly, using this solution configuration, the system provides ability to capture and process referrals defined by business Service Level Agreements (SLAs). For example, fully custom fax utility may be offered, which provides the ability to integrate with external fax vendors. Likewise, the solution may provide visibility, using custom dashboards, into incoming and outgoing communications via fax, email and phone using a proprietary algorithm for document management (e.g., splitting, merging, renaming, tagging, etc.)

Moreover, in accordance with disclosed embodiments, a patient onboarding dashboard may be provided to enable management of all relevant information including, for example, consent information, communication preferences and firewalled protected patient data. In particular, a firewalled and fully auditable approach may be implemented to protect patient data by leveraging advanced encryption schemes. Additionally, implementation of a proprietary case management approach to the overall patient journey enables the ability to capture the breadth of industry leading referral management business objectives. Furthermore, disclosed embodiments provide a solution that enables the ability to fully customize a patient care journey based on program specific work streams.

Further, as illustrated in connection with FIG. 1, the disclosed embodiments provide a custom-built solution offers a uniquely tailored approach for case management to manage the Patient Journey. In accordance with at least one implementation, each case rolls up to a patient and offers the hub a bird's eye view of the stage of treatment (‘Patient Journey’). In accordance with at least one implementation, each case acts as a driver for all patient related activities ranging from health benefits evaluation, prior authorization to shipment. In accordance with at least one implementation, the disclosed embodiments enable the case managers to track and complete the patient journey. In accordance with at least one implementation, multiple users may be able to access and update the same case at the same time. In accordance with at least one implementation, a progress bar may be provided that is fully customizable according to the work stream being enabled by disclosed embodiments.

Additionally, as illustrated in connection with FIG. 1, the disclosed embodiments provide improved functionality relating to task management and next best action planning. Disclosed embodiments provide a robust task management approach to aid in the patient journey. All configurable workflows can be tracked as tasks and activities. Additionally, disclosed embodiments may be pre-populated with a listing of recommended tasks based on industry knowledge. Examples include generating predictive tasks like Prior Authorization (PA) required or missing information based on the patient.

Furthermore, as illustrated in connection with FIG. 1, the disclosed embodiments provide improved analysis of Health Benefits Evaluation (HBE). The solution may drive a comprehensive workflow for benefits investigators and treatment initiation teams. Additionally, policy and coverage details for a patient may be integrated into task oriented dashboards and work streams for HBE teams with custom prioritization policies for HBE tasks. In accordance with at least one implementation, once a patient onboarding is complete integrated APIs enable HBEs to run eligibility at the click of a button and/or automatically. Patient coverage details can also be populated at the click of a button using API integrations. In accordance with at least one implementation, establishing coverage for a patient can also serve as a starting point for other stages such as Electronic Prescription or Referral (eRx), PA and Shipment. In accordance with at least one implementation, a task driven approach to benefits evaluation may be highly automated, wherein tasks with due dates such as ‘Fax a summary’ or ‘Contact Doctor’ may be generated once configured business rules are triggered. These tasks may also drive a separate work stream called workflows. Workflows can be utilized to just about configure any business rule. Simple rules such as changing a case category or complex ones involving multiple changes can be configured in a simplified manner. All outbound communications (faxes, calls, emails, etc.) can be generated as tasks. These may reside in a hub on which leaders can track and respond to the patient journey very effectively.

Moreover, as illustrated in connection with FIG. 1, the disclosed embodiments provide improved functionality to analyze and provide determinations regarding PA. In accordance with at least one implementation, a proprietary payor management module offers users the ability to track and manage claims and appeals. In accordance with at least one implementation, the disclosed system may be integrated with multiple partners to provide electronic PA services. In accordance with at least one implementation, the solution also offers capability to handle multiple payors for claims and appeals management. Disclosed embodiments can also be configured to support external initiatives such as Patient and co-pay assistance programs. In accordance with at least one implementation, an Electronic PA (‘ePA’) module may also integrate with Physician or Hospital Web Portals to meet industry standards. In accordance with at least one implementation, disclosed embodiments may enable prescribers to request PAs with the assistance of Case Managers.

Additionally, as illustrated in connection with FIG. 1, the disclosed embodiments provide improved analytics to support treatment planning. In accordance with at least one implementation, disclosed embodiments may offer case management for users to schedule and plan treatment external services. In accordance with at least one embodiment, a custom dashboard may provide ability to place pharmacy orders and track their shipment. In accordance with disclosed embodiments, automated triaging of pharmacy orders and tracking shipments. Treatment planning is also configurable as distinct service line. Intelligent algorithms may enable creating and updating of treatment cases in the treatment planning dashboard.

Moreover, as illustrated in connection with FIG. 1, the disclosed embodiments provide improved functionality for analyzing and determining patient adherence/closure. Disclosed embodiments may be configured to track a range of patient assistance programs. The data model is designed to be extensible to include a range of payors. Algorithms can identify patients whose shipments are due or overdue and appropriately alert users to ensure patient adherence. Integration with specialty pharmacies may be provided to enable offering real time insights into shipments. In accordance with disclosed embodiments, a task oriented approach may enable closure in a variety of workstreams, which may ensure no patient is left without a medicine, treatment or a follow up. Integration with pharmacy networks may enable efficient coordination of external services like home injection training or nurse scheduling.

Further, as illustrated in connection with FIG. 1, the disclosed embodiments provide improved reporting and analytics functionality for other aspects of the patient evaluation and treatment process to improve consistent quality of service and evaluation/treatment time efficiency. This may involve use of Key Performance Indicators (KPIs) to track metrics such as consent rate, paid shipped rate, turnaround times, patient adherence, etc. As a result, disclosed embodiments may have the ability to churn out standard reports as well offers the users the ability to configure custom reports. Integration adapters may be extended to support data warehouses and other external reporting systems.

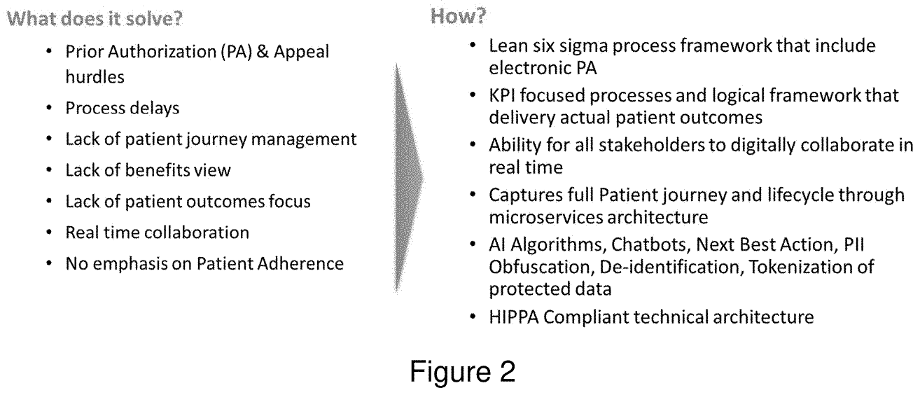

FIG. 2 provides an illustrative example of the technical utility provided by the disclosed embodiments and the underlying platform so as to highlight the technical problems to which the present innovation provides a technical solution. As shown in FIG. 2, the disclosed embodiments address current challenges in the market related to the overall Patient Journey. In particular, the disclosed embodiments enable a harmonized, lean, six sigma processes to overcome various PA and appeal hurdles. CareCloud also brings digital collaboration and full patient lifecycle enablement leveraging the microservices architecture to overcome the lack of comprehensive patient journey management.

In accordance with at least some disclosed embodiments, prescription services may be provided in a similar manner to the above described Health Benefits Evaluation utility. Thus, the disclosed embodiments may provide a cloud-implemented system and functionality that enables the customer to integrate with any service they desire for performing prescription evaluation for a patient. Accordingly, prescription evaluation may be implemented as a service that can provide details regarding the pharmacy, quantity and pricing of a drug approved and covered by the patient's current insurance plan.

Further, in accordance with at least one disclosed embodiment, prescription evaluation can be implemented in a cloud-based implementation using, for example, JavaScript Object Notation (JSON), which is a lightweight data-interchange format based on a subset of the JavaScript Programming Language Standard ECMA-262 3rd Edition—December 1999. For example, the functionality of the prescription evaluation may be implemented using the JSON code illustrated in Appendix A.

As is understood by one of ordinary skill in the art, JSON is a text format which is language independent. JSON uses conventions that are familiar to programmers of the C-family of languages, including C, C++, C #, Java, JavaScript, Perl, and Python. Such properties make JSON an effective data-interchange language.

The code included in Appendix A enables the customer to select the type of request and response handler they prefer to choose. This may also be limited sometimes based on the Service Provider's direction. Thus, the JSON code included in Appendix enables configuration of the API service the system may be using to perform prescription evaluation for a patient.

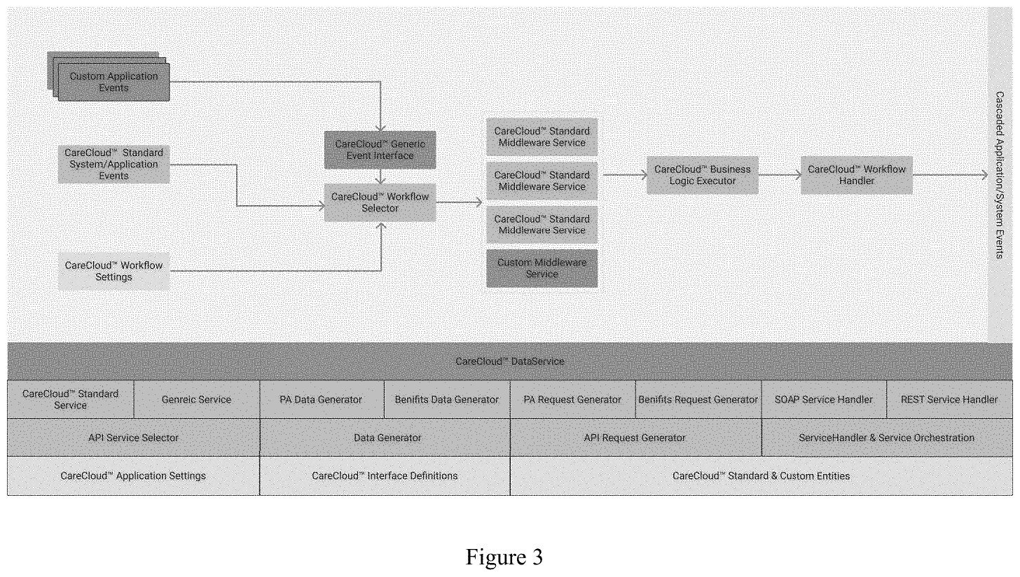

In accordance with at least some disclosed embodiments, task management, workflow management and next best action services may be provided in a similar manner to the above described Health Benefits Evaluation utility. Thus, the disclosed embodiments may provide a cloud-implemented system and functionality that enables task management, workflow management and next best action, as illustrated in FIG. 3. More specifically, a cloud-based implementation of the disclosed embodiments may offer a robust task management approach to aid in the patient journey. Thus, all configurable workflows can be tracked as tasks and activities in the solution. For example, the cloud-based implementation of the disclosed embodiments' algorithms may be used to leverage extensive industry knowledge to generate recommended tasks. Examples include generating predictive tasks like PA required or missing information based on the patient.

In accordance with at least some disclosed embodiments, prescription services may be provided in a similar manner to the above described Health Benefits Evaluation utility. Thus, the disclosed embodiments may provide a cloud-implemented system and functionality that enables workflow settings and process flow settings.

Therefore, disclosed embodiments may provide a cloud-based implementation that provides to ability to configure workflows and customize them based on the customer's needs. Workflows may be used to configure a set of tasks while processing the benefits for a patient. Workflows enable to Team Member(s) to perform certain tasks that are generated automatically by the system based on certain events that take place in the system.

Therefore, disclosed embodiments may provide a cloud-based implementation that enables these workflows events and workflows to be completely configured and customized using a Workflow Settings JSON file, an example of which being shown in Appendix B.

While processing the benefits for a patient, the evaluation may proceed through many stages in the system. Therefore, disclosed embodiments may provide a cloud-based implementation that enables the customer to customize these stages based on their requirements. Accordingly, each stage may be initiated and completed based on certain events that take place in the system. Therefore, disclosed embodiments may provide a cloud-based implementation in which these events and stages can be completely configured and customized using a process flow settings JSON file, an example of cod included in such a file being illustrated in Appendix C.

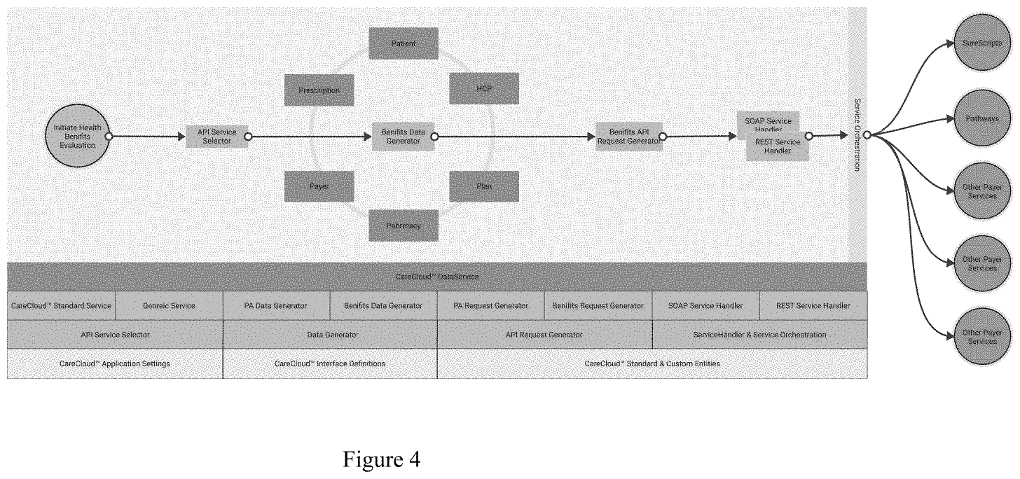

In accordance with at least some disclosed embodiments, the cloud-implemented system and functionality may enable a solution that drives a comprehensive workflow for benefits investigators and treatment initiation teams. Thus, as illustrated in FIG. 4, the solution may enable integration with industry leaders to obtain policy and coverage details for a patient. Further, task oriented dashboards and work streams may be provided for health benefits evaluation teams. Accordingly, disclosed embodiments provide a unique industry leading solution also offers custom prioritization policies for health benefits evaluation tasks.

Once a patient onboarding is complete, integrated APIs provided by disclosed embodiments can enable health benefits evaluations to perform patient eligibility functionality at the click of a button. Moreover, in accordance with at least some disclosed embodiments such functionality may be performed so as to check eligibility automatically.

Thus, patient coverage details can also be populated at the click of a button using API integrations provided in accordance with the disclosed embodiments. Establishing coverage for a patient can also serve as a starting point for other stages of process functionality provided by the disclosed embodiments such as PA and shipment. The unique task driven approach to benefits evaluation may be highly automated. Tasks with due dates such as ‘Fax a summary’ or ‘Contact Physician’ may be generated once configured business rules are triggered.

These tasks may also drive a separate work stream called workflows. Workflows can be utilized to configure virtually any business rule. Simple rules such as changing a case category or complex ones involving multiple changes can be configured just as simply. Thus, all, or virtually all outbound communications (faxes, calls, emails, etc.) can be generated as tasks, which can reside in a hub on which users can track and respond to the patient journey very effectively.

Further, disclosed embodiments may provide a cloud-based implementation that enables the customer to integrate with any service they desire for performing health benefits evaluation for a patient. Thus, health benefits evaluation as a service can provide details regarding the insurance policy and coverage details for a particular patient. For example, such health benefits evaluation functionality can be configured in a cloud-based implementation using, for example, the JSON code illustrated in Appendix D, which enables the customer to select the type of request and response handler they prefer to choose. This may also be limited sometimes based on the Service Provider. This piece of JSON may also enable configuration of the API service the system will be using to perform health benefits evaluation for a patient.

In accordance with at least some disclosed embodiments, PA services may be provided in a similar manner to the above described health benefits evaluation utility. Thus, the disclosed embodiments may provide a cloud-implemented system and functionality that enables proprietary payor management module that offers users the ability to track and manage claims and appeals. Disclosed embodiments may enable integration with multiple partners to provide electronic PA services. As such, the disclosed solution also offers capability to handle multiple payors for claims and appeals management as well as offering the ability to support external assistance programs such as patient assistance and co-pay.

In accordance with disclosed embodiments, an ePA module also integrates with Physician portals to meet industry standards. Thus, the cloud-based implementation of the disclosed embodiments enables prescribers to request PAs with the assistance of case managers.

Further, in accordance with disclosed embodiments, the cloud-based implementation may enable the customer to integrate with any service they desire for performing PA evaluation for a patient. For example, PA evaluation can be configured in a cloud-based implementation using the JSON code shown in Appendix E. That code enables the customer to select the type of request and response handler they prefer to choose. This may also be limited sometimes based on the service provider. This piece of JSON also enables to configure the API service the system will be using to perform PA evaluation for a patient.

In accordance with at least some disclosed embodiments, treatment planning (for example, using a nurse network) may be provided. Thus, the disclosed embodiments may provide a cloud-implemented system and functionality that enables case management for users to schedule and plan treatment external services. The disclosed custom dashboard provides the ability to place pharmacy orders and track their shipment. Moreover, there is the ability to leverage a custom algorithm that automates triaging pharmacy orders and tracking shipments. Treatment planning may also be configurable as a distinct service line in the cloud-based implementation of the disclosed embodiments. Intelligent algorithms may also enable creating and updating of treatment cases in the treatment planning dashboard.

In accordance with at least some disclosed embodiments, patient adherence and closure services may be provided. More specifically, the cloud-based implementation of the disclosed embodiments can be configured to track a range of patient assistance programs. The data model is designed to be extensible to include a range of payors. Algorithms can identify patients whose shipments are due or overdue and appropriately alert users to ensure patient adherence. Integration with specialty pharmacies enables the cloud-based implementation of the disclosed embodiments to offer real time insights into shipments.

The cloud-based implementation of the disclosed embodiments also provides a task oriented approach to closure that is critical in a variety of workstreams. This ensures no patient is left without a drug or a follow up. Integration with pharmacy networks also enables the disclosed embodiments to enable users to efficiently coordinate external services like home injection training or nurse scheduling.

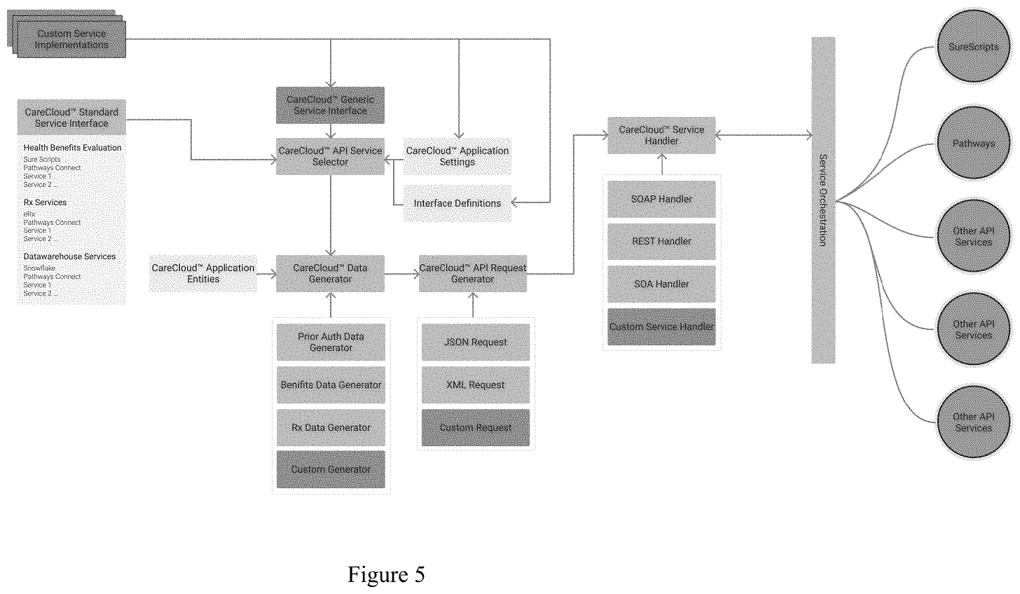

In accordance with at least some disclosed embodiments, reporting, analytics and data services may be provided and are shown in connection with FIG. 5. As such, it should be understood that the disclosed embodiments include use of proprietary KPIs to track the following metrics: consent rate, paid shipped rate, turnaround times, and patient adherence. The cloud-based implementation of the disclosed embodiments' algorithms have the ability to churn out standard reports as well offers the users the ability to configure custom reports. Integration adapters in the disclosed embodiments can be extended to support data warehouses and other external reporting systems.

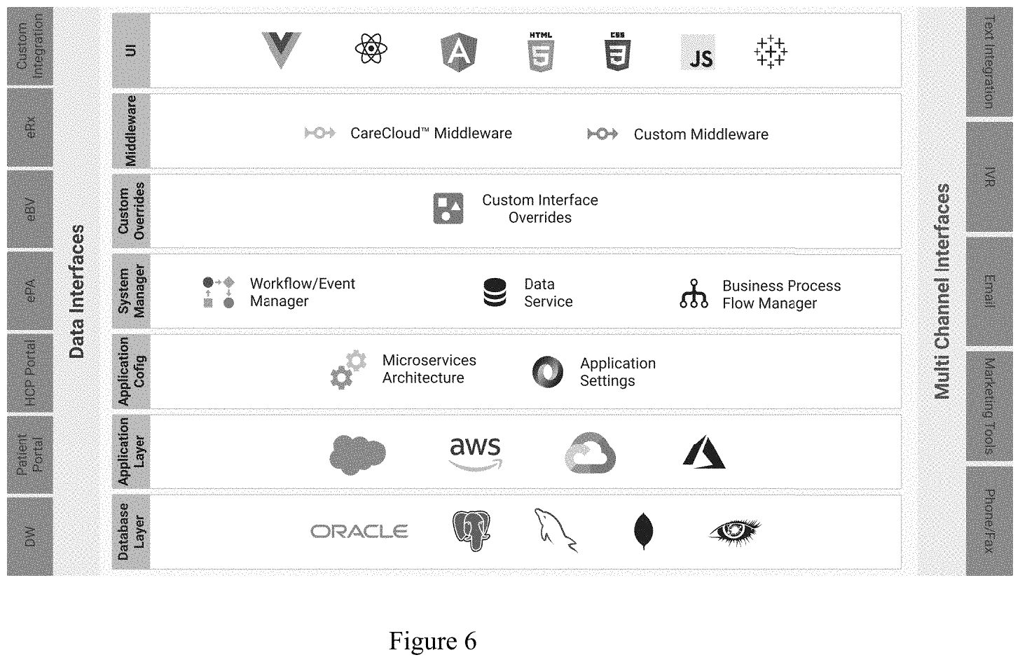

FIG. 6 provides an illustrative example of the architecture of the cloud-based system. The architecture provides an outline of data integrations and interfaces that are configurable in accordance with the disclosed embodiments. It also showcases the wide array of software and platform compatibility across the software stack—from databases to user interfaces. The extensible middleware layer offers greater flexibility for custom business outcome focused implementations. A robust microservices layer enables scalable overrides and configurable business critical workflows.

It should be understood that, with regard to configuration files, the application settings file may have all the details for configuring cloud-based implementation of the disclosed embodiments. In particular, that file may contain application details, product branding, service handlers' API services used for each of the services and response data mappings. For example, application details define the name of the product, current version, customer details, the logo and other assets being used by for the cloud-based implementation. These assets may be configurable and can be customized using the application settings file.

Thus, it should be understood that this file enables the customer to configure the type of service request they prefer using for the API requests and responses from the system. A few examples of these requests are SOAP, REST etc. Further API services being used for the cloud-based implementation may be configured using the Application Settings file. Accordingly, the different API services that can be configured may include benefits evaluation, PA, prescription evaluation and any other services that may be required. The customer can define the names of the services being used, current version, URL, authentication details and tokens and any other details that may be required. This enables the disclosed embodiments to be independent of any service and highly customizable.

The data generator being used for the disclosed embodiments may enable the system to convert the JSON response received into data that can be processed and stored in a cloud-based implementation entities. This file enables to configure different classes, source and target entity mappings and any other data processing logics that may be required. An example of an application settings file is provided in Appendix F.

In accordance with disclosed embodiments an interface settings file may have each of the API services configured and mappings used to load to the response data into the disclosed entities. For each API service, the customer may configure the service details, end points, authentication and the source and target data mappings. Service details may define name of the service, type of API call used, URL, current version and any other details that may be required. This can be defined for each API Service separately and may enable the disclosed embodiments to be highly customizable and configurable. End points may be used to define the addresses used for sending and fetching data, to and from the API service being used. Authentication may define the authentication codes and tokens used to verify the credentials while sending and fetching data, to and from the API service. Source and target mappings may be used to define how the incoming data will be mapped and stored in a cloud-based implementation entities. The data generator class may use the mappings configured in a cloud-based implementation to process the data. Mappings can also be used to configure any transformations and other actions required before storing the data in a cloud-based implementation entities. An example, of an interface settings file is illustrated in Appendix G.

In accordance with disclosed embodiments, a workflow settings file may have each of the workflow actions configured based on events affecting the cloud-based implementation for each entity. More specifically, for each entity, a workflow settings file may enable the customer to configure each event to generate activities and actionable tasks along with due dates and actions required. Each event can be used to define the event details, workflow details, and the affected cloud-based implementation for entities.

Event details may define the name, type, description, actions required and any other configuration that may be required. Workflow details may be used to define the workflow actions required, name, due date, priority, assignment, entities affected and any other details that may be required. Appendix H includes an example of a sample workflow settings file.

A process flow settings file may include each of the system process flow actions configured based on events affecting the cloud-based implementation for each business process. For each event, process flow settings can be used to define the event name, type, description, system conditions and state, target actions required to set the current stage of the disclosed embodiments. Appendix I includes an example of a process flow settings file. Fax processing dashboard.

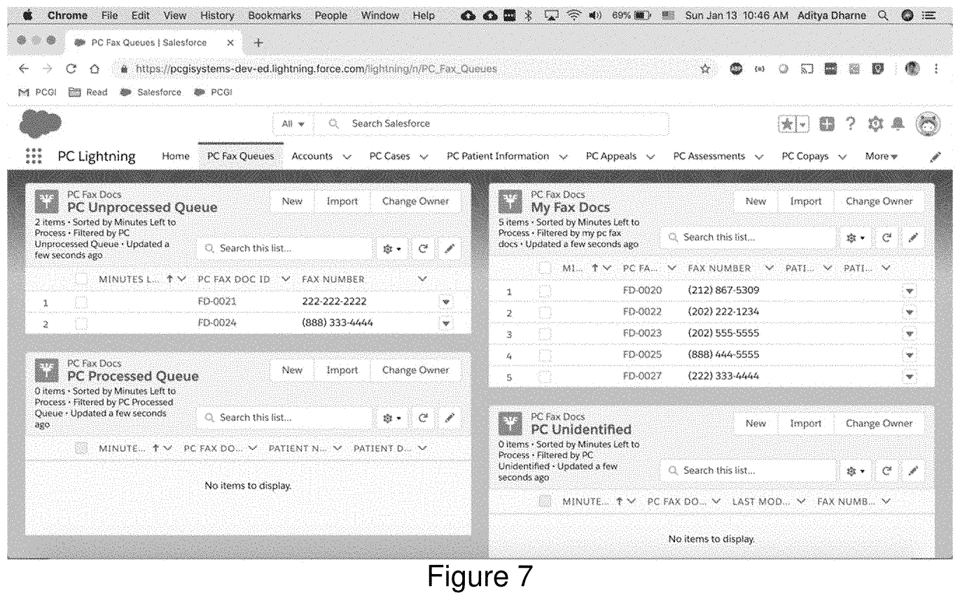

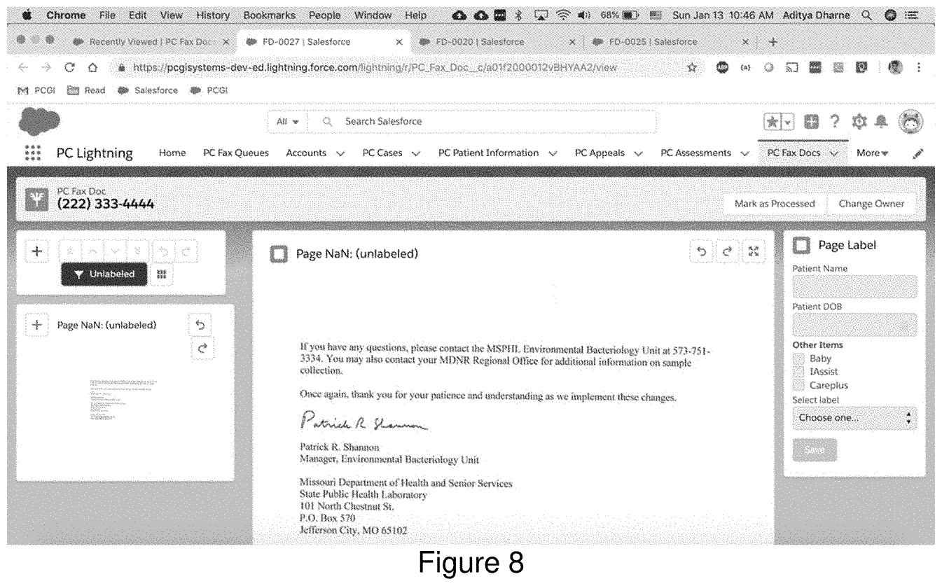

FIG. 7 illustrates an example a fax processing dashboard provided in accordance with the disclosed embodiments. Likewise, FIG. 8 illustrates an example of a fax processing utility user interface.

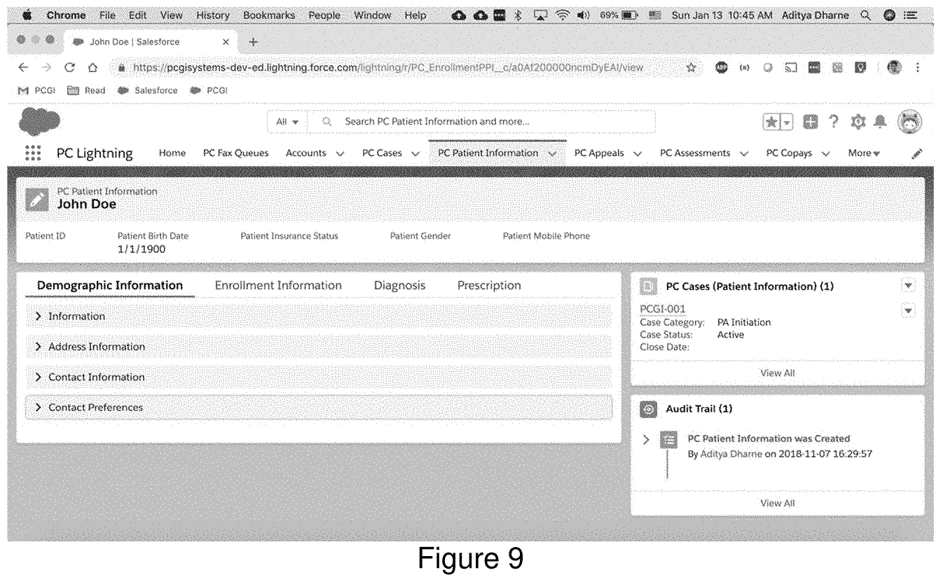

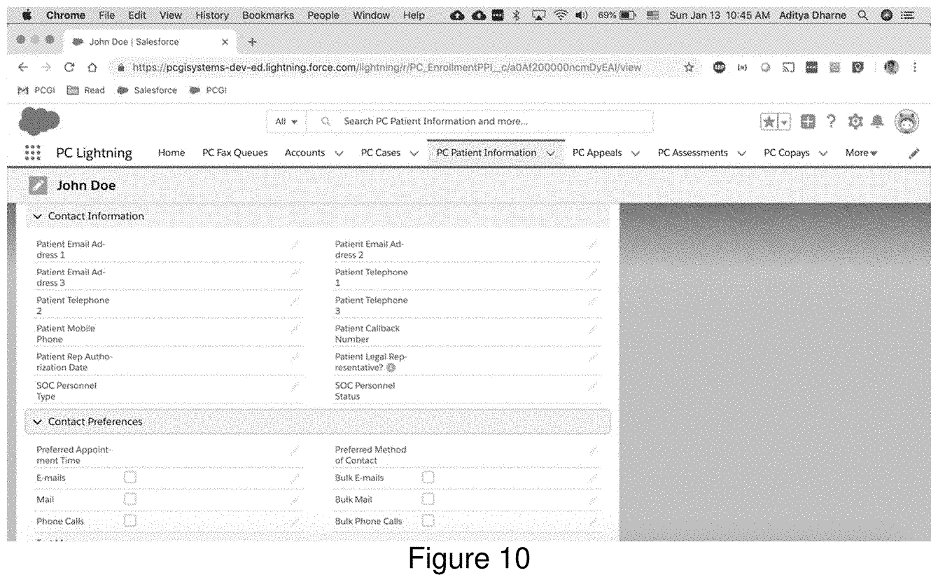

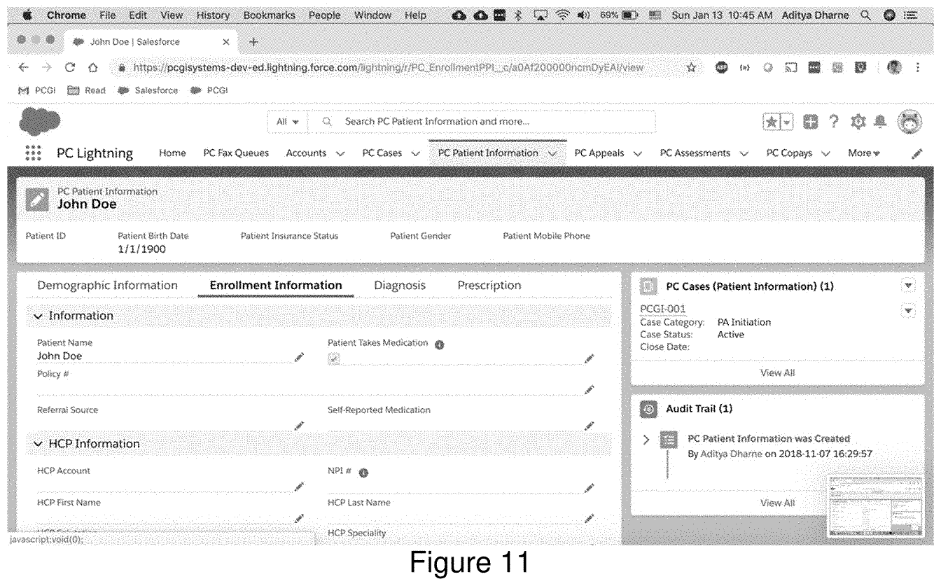

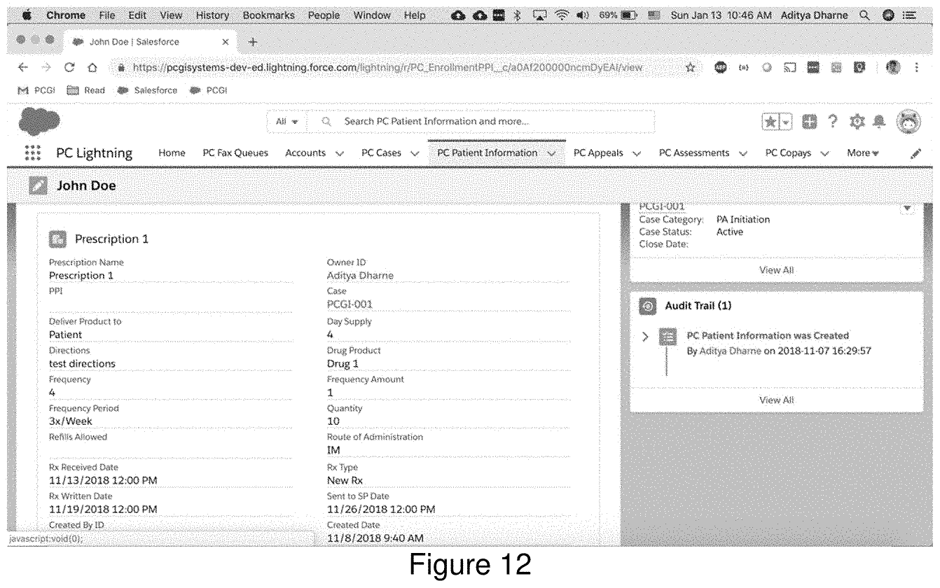

FIGS. 8-11 illustrate examples of patient view user interface screens included in a patient dashboard configured to enable input of various data associated with a patient and review of such and related information accessed via that input.

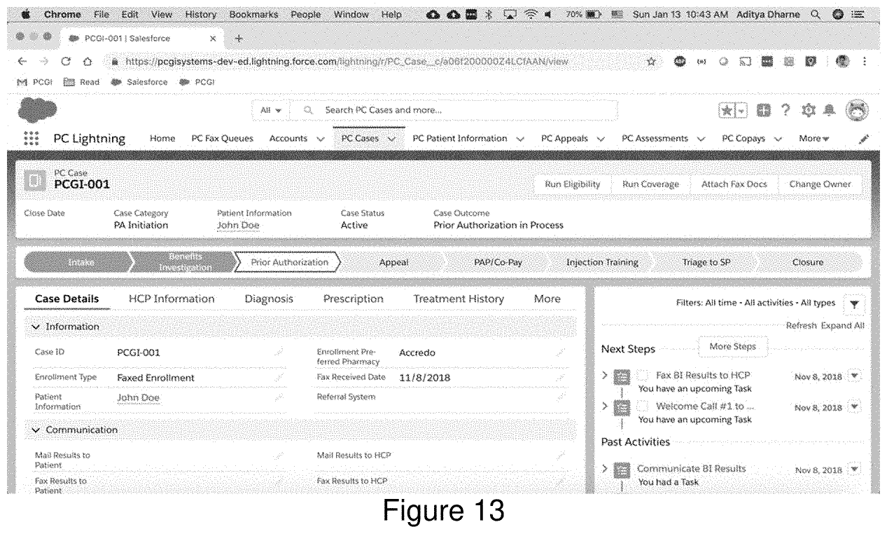

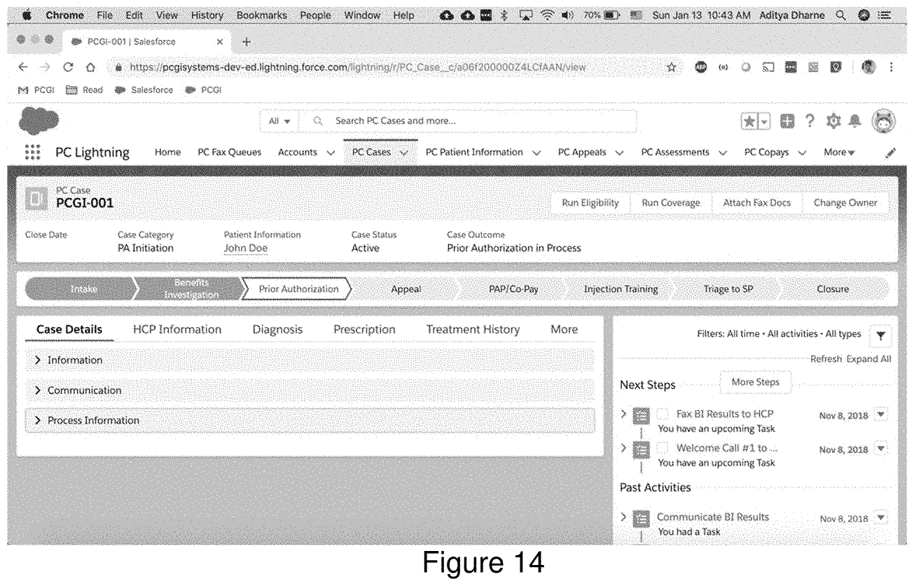

FIGS. 13-14 illustrate examples of case view user interface screens included in a case view dashboard configured to enable input of various data associated with a particular case and review of such and related information accessed via that input.

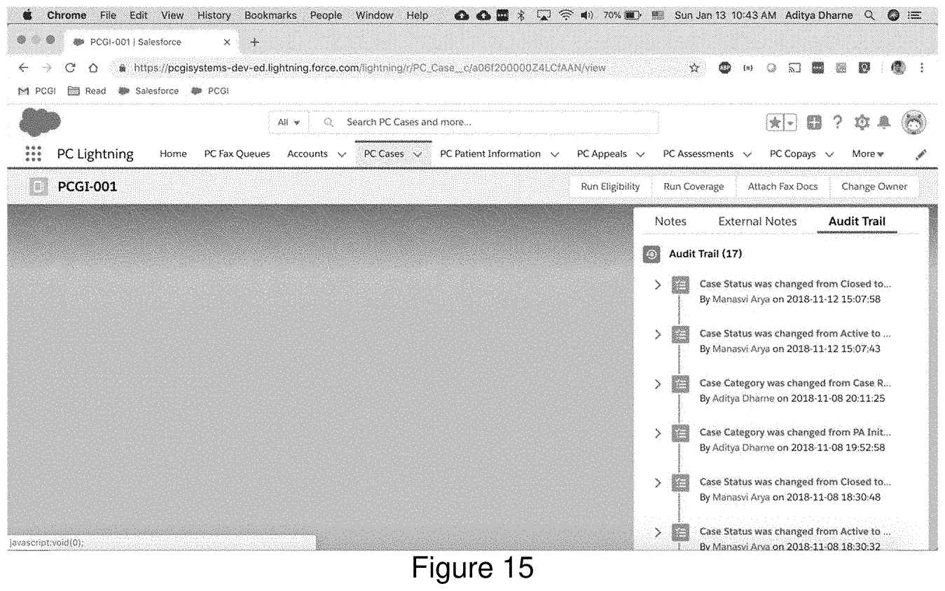

FIG. 15 illustrate an example of an audit trail on case dashboard configured to enable input of various data associated with a patient's case and review of such and related information accessed via that input.

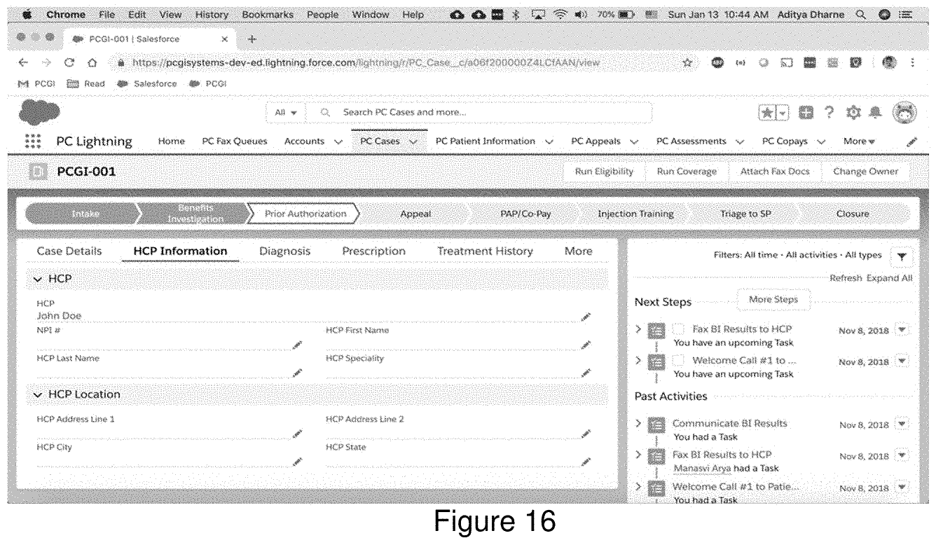

FIG. 16 illustrate an example of a Health Care Professional (or Provider (HCP) Information on Case dashboard configured to enable input of various data associated with health care provider(s) on a particular case and review of such and related information accessed via that input. Note, in at least one implementation, such a dashboard may be integrated with commercially available technology such as that provided by Veeva™ and/or Master Data Management (MDM) systems.

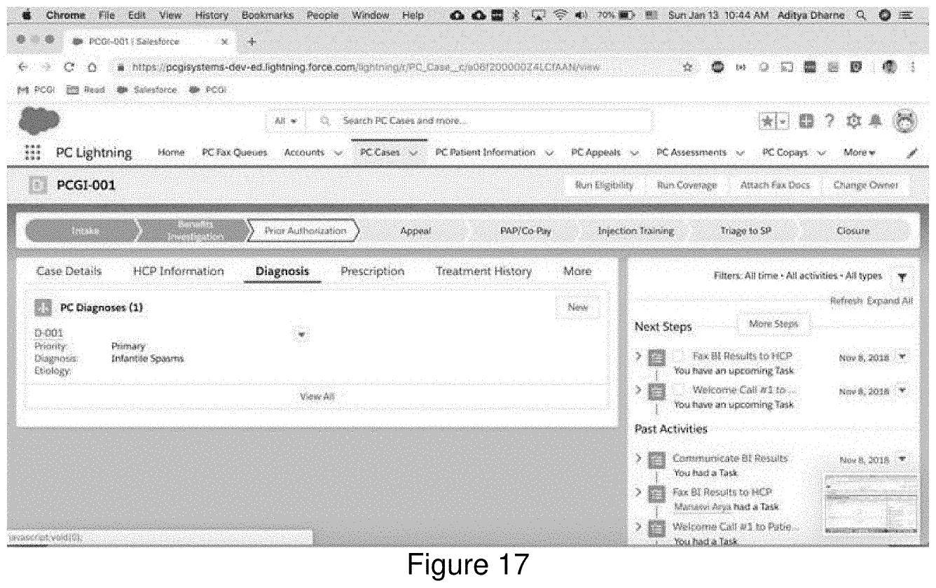

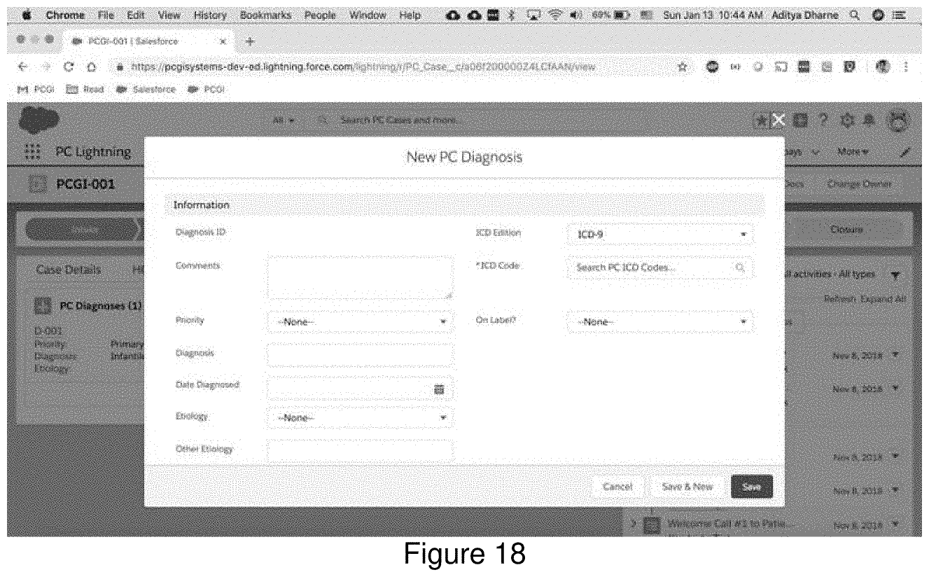

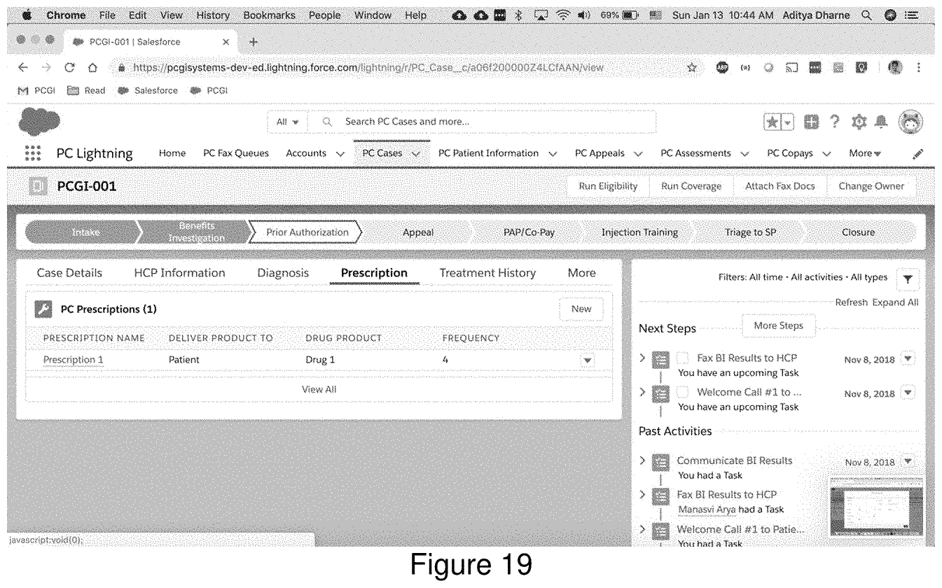

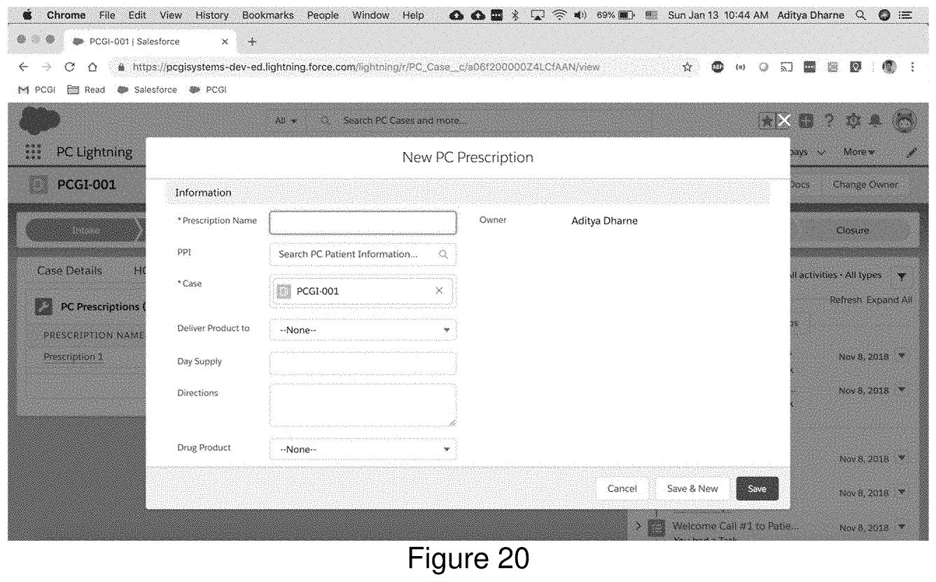

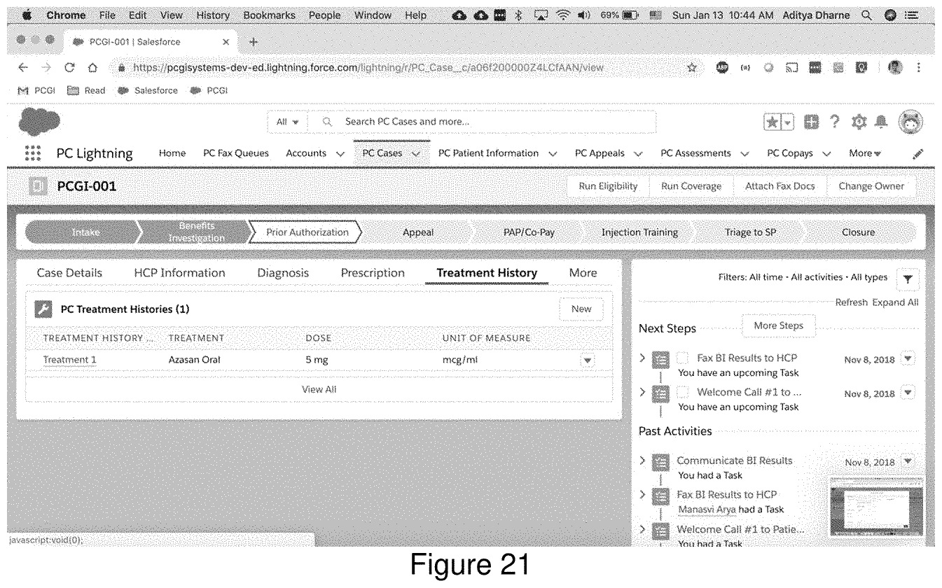

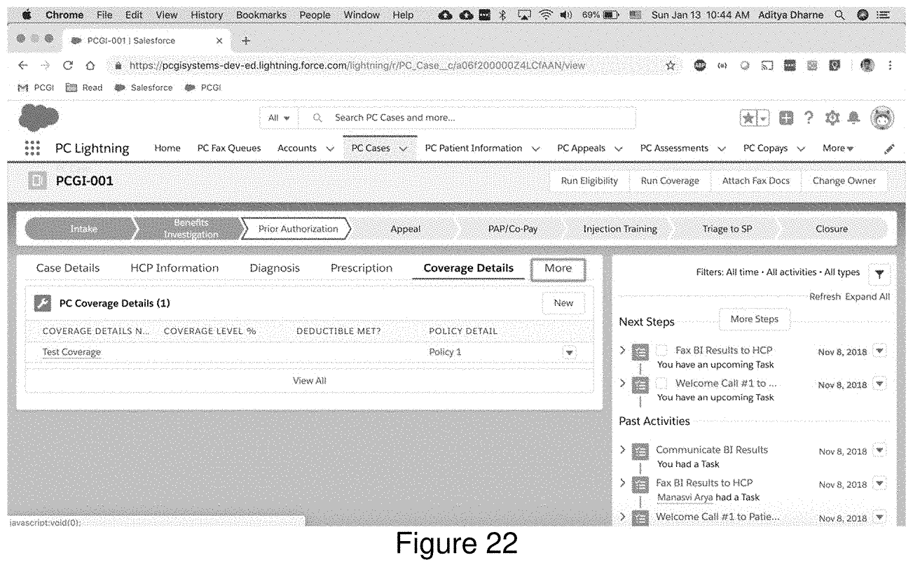

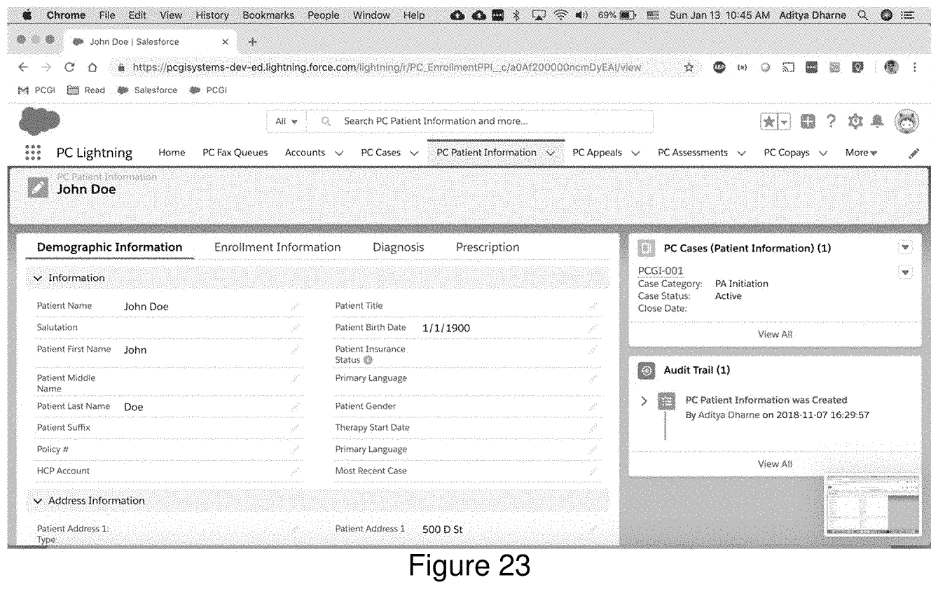

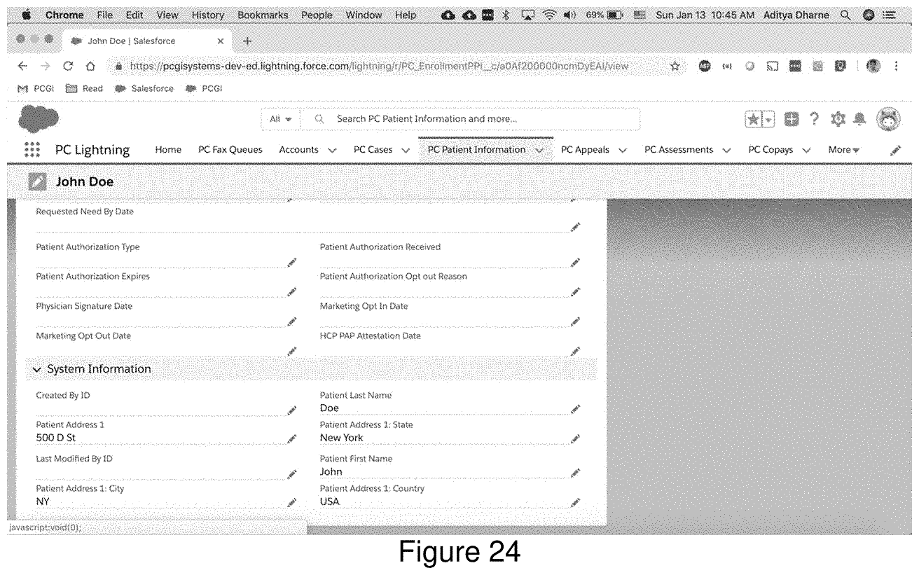

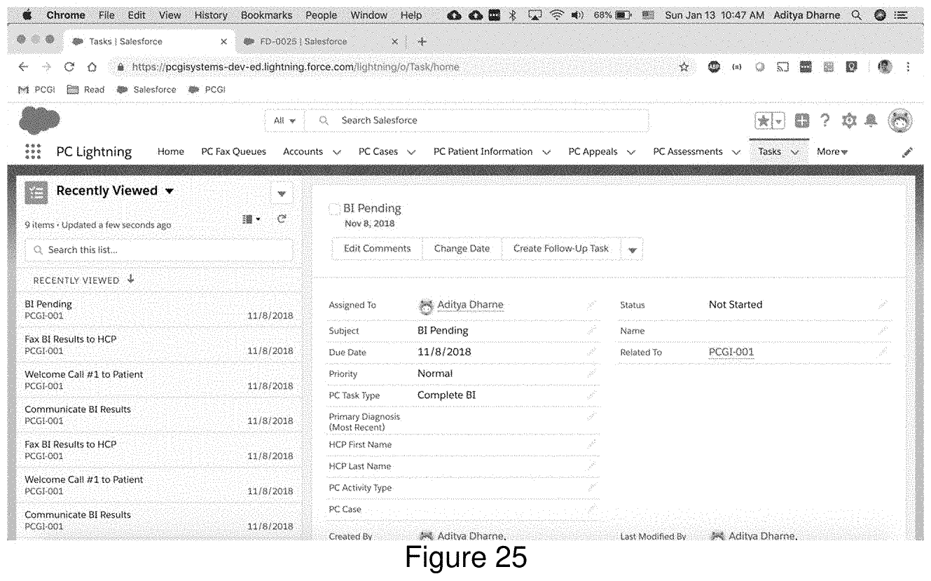

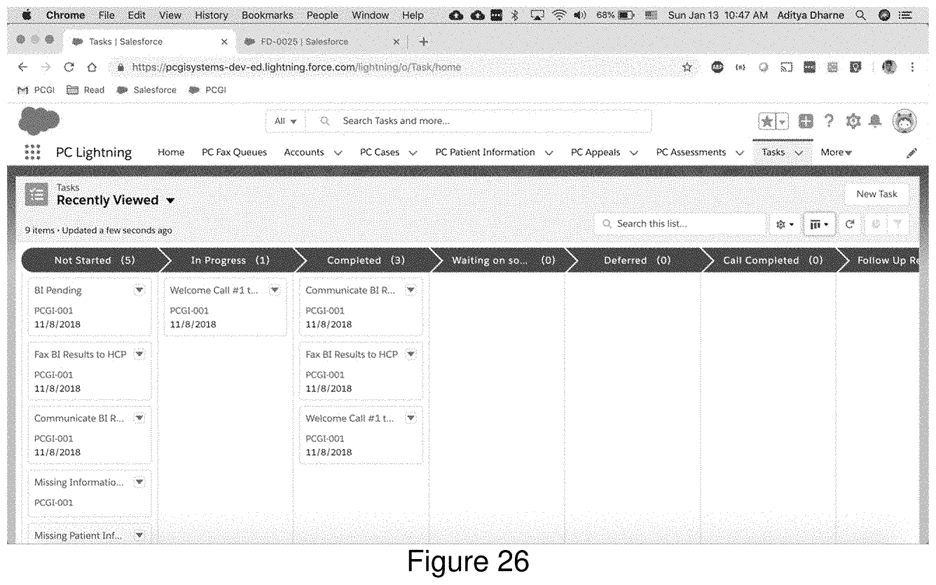

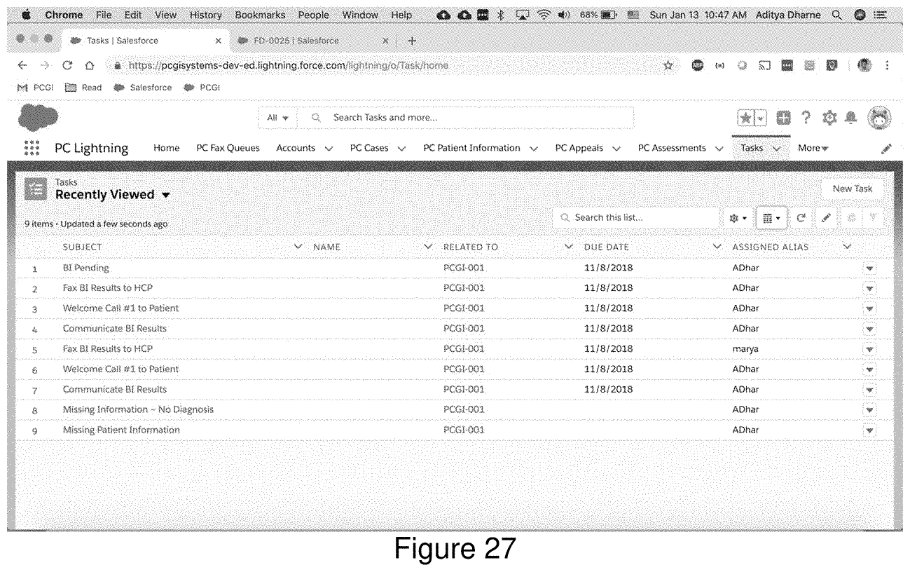

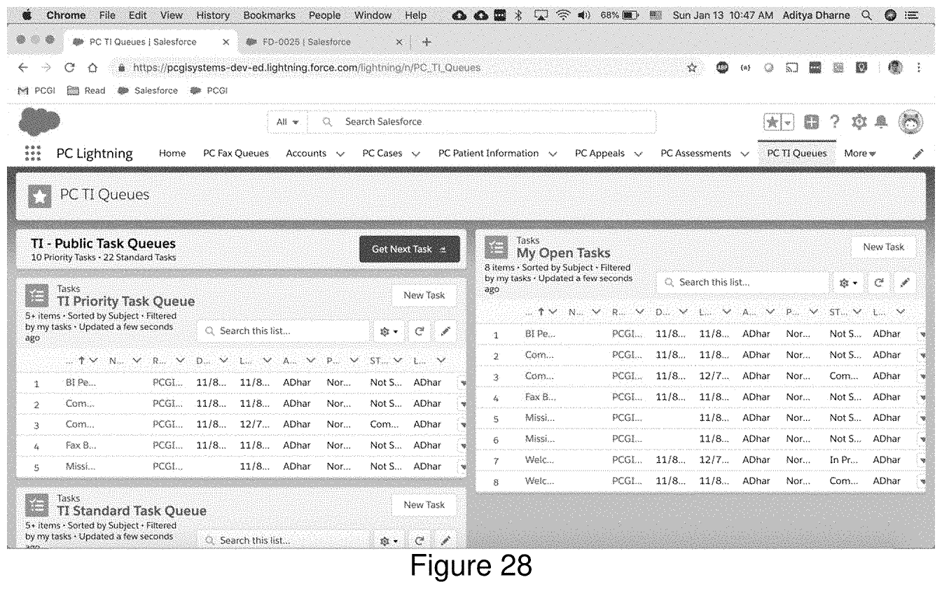

Moreover, FIGS. 17-28 illustrate examples of various user interfaces configured to enable input of various data associated with individual components of a patient's case and review of such and related information accessed via that input. FIG. 17 illustrates an exemplary screen shot of user interface in the system wherein cases are listed for access of information. FIG. 18 illustrates an exemplary screen shot of a user interface in the system wherein information for a new case (diagnosis) may be input and saved in accordance with various disclosed embodiments. FIG. 19 illustrates an exemplary screen shot of a user interface in the system wherein prescriptions are listed for access of information. FIG. 20 illustrates an exemplary screen shot of a user interface in the system wherein information for a new prescription may be input and saved in accordance with various disclosed embodiments. FIG. 21 illustrates an exemplary screen shot of a user interface in the system wherein treatment history information is listed for access of information. FIG. 22 illustrates an exemplary screen shot of a user interface in the system wherein coverage information is listed for access of information. FIGS. 23-24 illustrate exemplary screen shot of user interfaces in the system wherein patient demographic and system information is listed for access of information. FIGS. 25-28 illustrate exemplary screen shots of user interfaces in the system for managing task and workflows.

In order for the disclosed embodiments to provide functionality to perform various above-described customer, i.e., patient related functionality implemented in a cloud computing environment, and to implement the above-described front end that includes a user interface that includes various web-based screens providing input and/or output functionality to a user for inputting and/or reviewing output data regarding one or more of the above-identified patient related functionality, a backend architecture is provided that supports providing the user interfaces in a cloud implemented environment.

Thus, in accordance with the disclosed embodiments, one or more of the subcomponents of the system may be implemented on one or more servers coupled to one or more public and/or private communication networks, e.g., the Internet. As a result, it should be appreciated that the functionality disclosed herein and interaction with the system subcomponents may be accessed via one or more computers, e.g., a mobile device such as a mobile smartphone, a PDA, a personal computer, a multimedia computer, a laptop, and the like. Additionally, or alternatively, the functionality disclosed herein may be implemented in whole or in part as a software application, e.g., a mobile application, which is a software program or application designed to run on computers such as smartphones, tablet computers and other mobile devices.

It should be understood that the software used to implement the disclosed embodiments may communicate with one or more databases running on such servers to provide to enable storage of uploaded data from candidates as well as storage of datasets for download to candidates as part of the skills testing subcomponent one or more communication networks. Such networks may include any type of communication network, such as dedicated public or private wired networks, WI-FI, a cellular communication network, including but not limited to a second Generation (2G) network, a 2.5 Generation network, a third Generation (3G) network utilizing Global System for Mobile Communications (GSM), Wideband Code Division Multiplex Access (WCDMA), Code Division Multiplex Access (CDMA), or Time Division Multiplex Access (TDMA), General Packet Radio Services (GPRS), Universal Mobile Telephone System (UMTS). Additionally, such networks can also be implemented as a combination of two or more technologies i.e., a hybrid network. Further, the network(s) may also include generic Internet access using any transport methods.

The functionality disclosed herein may be implemented in various configurations using computer-readable media for carrying or having computer-executable instructions or data structures stored thereon. Such computer-readable media can be any available media that can be accessed by a general purpose or special purpose computer. By way of example, and not limitation, such computer-readable media can comprise physical storage and/or memory media such as RAM, ROM, EEPROM, CD-ROM or other optical disk storage, magnetic disk storage or other magnetic storage devices, or any other medium which can be used to carry or store desired program code means in the form of computer-executable instructions or data structures and which can be accessed by a general purpose or special purpose computer. Computer-executable instructions comprise, for example, instructions and data which cause a general purpose computer, special purpose computer, or special purpose processing device to perform a certain function or group of functions.

Accordingly, although not illustrated, it should be understood that the functionality may be implemented using a server that provides or includes a processor connected to a user interface, computer readable memory and/or other data storage and a display and/or other output device. Computer executable instructions and data used by a processor may be stored in the computer readable memory included in the server or implemented with any combination of read only memory modules or random access memory modules, optionally including both volatile and nonvolatile memory.

While this invention has been described in conjunction with the specific embodiments outlined above, it is evident that many alternatives, modifications and variations will be apparent to those skilled in the art. Accordingly, the various embodiments of the invention, as set forth above, are intended to be illustrative, not limiting. Various changes may be made without departing from the spirit and scope of the invention.

Further, it should be understood that the functionality described in connection with various described subcomponents of various invention embodiments may be combined or separated from one another in such a way that the architecture of the invention is somewhat different than what is expressly disclosed herein. Moreover, it should be understood that, unless otherwise specified, there is no essential requirement that methodology operations be performed in the illustrated order; therefore, one of ordinary skill in the art would recognize that some operations may be performed in one or more alternative order and/or simultaneously.

Although certain illustrative embodiments have been described in detail above, variations and modifications exist within the scope and spirit of this disclosure as described and as defined in the claims included in this application.

APPENDIX A

```
“Service Handler” : {
    “Handler Name” : “REST/SOAP/SOA/.......”,
    “Handler Class” : “ ”,
    “Handler Override Class” : “ ”,
    “Handler Class Version” : “ ”,
    “Allow OTA” : “Yes”,
    “Version”. “v1.....”
  }
“API Service” ; {
    “Rx” : {
      “Service Name” : “Pathways/.......”,
      “Version” : “v1”,
      “Allow OTA” : “Yes”
      “Base URL” : “”;
      “Authentication” : {
      “Types” : “ ”,
      “AUTH URL” : “ ”
      }
    }
}
```

APPENDIX B

```
{
  “Events”: [
  {
    “Event Name/Title” : “XYZ”,
    “Type” : “ABC”,
    “Description” : “....”,
    “System Action” : “.....”,
    “System Conditions” : {
      “Condition 1”: “.....”;
      “Condition n” : “.....”;
    }
    “Process Stage” : “Office Visit/Diagnosis”;
    “Child Stage” : “Null”,
    “Grand Child Stage” : “Null”,
    “Target Actions: {
      “ID” : “..........”,
      “Type” : “........”,
      “Name” : “........”
      “Description” : “.......”
    }
  }
```

APPENDIX C

```
{
  “Events” : [
  {
    “Event Name/Title” : “XYZ”,
    “Type” : “ABC”,
    “Description” : “....”,
    “System Action” : “.....”,
    “System Conditions” : {
      “Condition 1” : “.....”;
      “Condition n” : “.....”;
    }
    “Stage” : {
    “Parent Stage” : “Office Visit/Diagnosis”;
    “Child Stage” : “Null”,
    “Grand Child Stage” : “Null”,
    }
    “Target Actions: {
      “ID” : “..........”,
      “Type” : “........”,
      “Name” : “........”
      “Description” : “.......”}
```

APPENDIX D

```
“Service Handler” : {
    “Handler Name” : “REST/SOAP/SOA/.......”,
    “Handler Class” : “ ”,
    “Handler Override Class” : “ ”,
    “Handler Class Version” : “ ”,
    “Allow OTA”. “Yes”,
    “Version”. “v1.....”
  }
“API Service” : {
    “Benefits” : {
      “Service Name” : “Pathways/.......”,
      “Version” : “v1”,
      “Allow OTA”: “Yes”
      “Base URL”: “”;
      “Authentication” : {
      “Types” : “ ”,
      “AUTH URL” : “ ”
      }
    }
}
```

APPENDIX E

```
“Service Handler” : {
    “Handler Name” : “REST/SOAP/SOA/.......”,
    “Handler Class” : “ ”,
    “Handler Override Class” : “ ”,
    “Handler Class Version” : “ ”,
    “Allow OTA”. “Yes”,
    “Version”. “v1.....”
  }
“API Service” : {
    “Prior Auth” : {
      “Service Name” : “Pathways/.......”,
      “Version” : “v1”,
      “Allow OTA” : “Yes”
      “Base URL” : “”;
      “Authentication” : {
      “Types” : “ ”,
      “AUTH URL” : “ ”
      }
    }
}
```

APPENDIX F

```
{
    “Application” : {
      “Name” : “Therefore, disclosed embodiments may
provide a cloud-based implementation that”,
      “Customer/Client” : “XYZ”,
      “Version” : “v1.....”,
      “Allow OTA” : “Yes”,
      “Host” : “......”,
      “Platform” : {
          “OS” : “Windows/IOS”,
          “Base System” : “Salesforce/Django”,
          .
          .
          .
      }
      “Logo/Branding” : {
      “Asset 1” : “......”,
      “Asset 2” : “......”,
      “Asset n” : “......”
      }
  }
    “Service Handler” : {
      “Handler Name”. “REST/SOAP/SOA/.......”,
      “Handler Class” : “ ”,
      “Handler Override Class” : “ ”,
      “Handler Class Version” : “ ”,
      “Allow OTA” : “Yes”,
      “Version”: “v1.....”
    }
    “API Service” : {
      “Rx” : {
          “Service Name” : “Pathways/.......”,
          “Version” : “v1”,
          “Allow OTA” : “Yes”
          “Base URL” : “”;
          “Authentication” : {
          “Types” : “ ”,
          “AUTH URL” : “ ”
          }
      }
      “Benefits” : {
          “Service Name” : “Pathways/.......”,
          “Version” : “v1”,
          “Allow OTA” : “Yes”
          “Base URL” : “”;
          “Authentication” : {
          “Types” : “ ”,
          “AUTH URL” : “ ”
          }
      }
      “Prior Auth” : {
          “Service Name” : “Pathways/.......”,
          “Version” : “v1”,
          “Allow OTA” : “Yes”
          “Base URL” : “”;
          “Authentication” : {
          “Types” : “ ”,
          “AUTH URL” : “ ”
          }
      }
    }
    “Data Generator” : {
      “Rx” : {
          “Object1/Table1” : “Prescription”,
          “Object2/Table2” : “....”,
          “Class1” : “DataGen”,
          “Class2” : “......”
          .
          .
          “Mappings” : [
          {
          “Source Entity” : “ABC”,
          “Target Entity” : “XYZ,
          “Mapping” : [
          “Field 1” : {
            “Source Fields” : ,
            “Target Field” : ,
            “Transformations” : {
            “Name” : ,
            “Logic” :
            }
          }
          “Field 2” : {
            “Source Fields” : ,
            “Target Field” : ,
            “Transformations” : {
            “Name” : ,
            “Logic” :
            }
          }]
        }
          {
          “Source Entity” : “ABC”,
          “Target Entity” : “XYZ,
          “Mapping” : [{
            “Source Fields” : ,
            “Target Field” : ,
            “Transformations” : {
            “Name” : ,
            “Logic” :
            }
          }]
        }]
      }
      “Benefits“ : {
          “Object1/Table1” : “Prescription”,
          “Object2/Table2” : “....”,
          “Class1” : “DataGen”,
          “Class2” : “......”
          .
          .
          .
          .
          .
          “Mappings” : [
          “Source Entity”: “ABC”,
          “Target Entity” : “XYZ,
          “Mapping” : [
          “Field 1” : {
            “Source Fields” : ,
            “Target Field” : ,
            “Transformations” : {
            “Name” : ,
            “Logic” :
            }
          }
          “Field 2” : {
            “Source Fields” : ,
            “Target Field” : ,
            “Transformations” : {
            “Name” : ,
            “Logic” :
            }
          }]
        }
          {
          “Source Entity” : “ABC”,
          “Target Entity” : “XYZ,
          “Mapping” : [{
            “Source Fields” : ,
            “Target Field” : ,
            “Transformations” : {
            “Name” : ,
            “Logic” :
            }
          }]
        }]
      }
      “Prior Auth” : {
          “Object1/Table1”: “Prescription”,
          “Object2/Table2” : “....”,
          “Class1” : “DataGen”,
          “Class2” : “......”
          .
          .
          .
          .
          .
          “Mappings” : [
          {
          “Source Entity”: “ABC”,
          “Target Entity” : “XYZ,
          “Mapping” : [
          “Field 1” : {
            “Source Fields” : ,
            “Target Field” : ,
            “Transformations” : {
            “Name” : ,
            “Logic” : ,
            }
          }
          “Field 2” : {
            “Source Fields” : ,
            “Target Field” : ,
            “Transformations” : {
            “Name” : ,
            “Logic” :
            }
          }]
        }
          {
          “Source Entity” : “ABC”,
          “Target Entity” : “XYZ,
          “Mapping” : [{
            “Source Fields” : ,
            “Target Field” : ,
            “Transformations” : {
            “Name” : ,
            “Logic” :
            }
          }]
        }]
      }
    }
```

APPENDIX G

```
{
  “Rx” : {
    “ID” : “.....”,
    “Service Name” : “Pathways/.......”,
    “API” : “REST/SOAP/SOA/.......”,
    “URL” : “http://xyz.com”,
    “WSDL” :
    “EndPoints” : {
        “Get” : “http://xyz.com”,
        “Post” : “http://xyz.com”
    }
    “Authentication” : “xyz”,
    “Data Load Mappings” : [
        {
        “Source Entity” : “ABC”,
        “Target Entity” : “XYZ,
        “Mapping” : [
        “Field 1” : {
          “Source Fields” : “PatientID”,
          “Target Field” : “Therefore, disclosed embodiments
may provide a cloud-based implementation thatPatient
Number/Custom Field”,
          “Transformations” : {
          “Name” : ,
          “Logic” :
          }
        }
        “Field 2” : {
          “Source Fields” : ,
          “Target Field” : ,
          “Transformations” : {
          “Name” : ,
          “Logic” :
          }
        }]
      }
        {
        “Source Entity”: “ABC”,
        “Target Entity” : “XYZ,
        “Mapping” : [{
          “Source Fields” : ,
          “Target Field” : ,
          “Transformations” : {
          “Name” : ,
          “Logic” :
          }
        }]
      }]
  }
  “Benefits” : {
    “ID” : “.....”,
    “Service Name” : “Pathways/.......”,
    “API” : “REST/SOAP/SOA/.......”,
    “URL” : “http://xyz.com”,
    “EndPoints” : {
        “Get”: “http://xyz.com”,
        “Post” : “http://xyz.com”
    }
    “Authentication” : “xyz”,
    “Data Load Mappings” : [
        {
        “Source Entity” : “ABC”,
        “Target Entity” : “XYZ,
        “Mapping” : [
        “Field 1” : {
          “Source Fields” : “PatientID”,
          “Target Field” : “Therefore, disclosed embodiments
may provide a cloud-based implementation thatPatient
Number/Custom Field”,
          “Transformations” : {
          “Name” : ,
          “Logic” :
          }
        }
        “Field 2” : {
          “Source Fields” : ,
          “Target Field” : ,
          “Transformations” : {
          “Name” : ,
          “Logic” :
          }
        }]
      }
        {
        “Source Entity” : “ABC”,
        “Target Entity” : “XYZ,
        “Mapping” : [{
          “Source Fields” : ,
          “Target Field” : ,
          “Transformations” : {
          “Name” : ,
          “Logic” :
          }
        }]
      }]
  }
  “Prior Auth” : {
    “ID” : “.....”,
    “Service Name” : “Pathways/.......”,
    “API”. “REST/SOAP/SOA/.......”,
    “URL”: “http://xyz.com”,
    “EndPoints” : {
        “Get” : “http://xyz.com”,
        “Post” : “http://xyz.com”
    }
    “Authentication”. “xyz”,
    “Data Load Mappings” : [
        {
        “Source Entity” : “ABC”,
        “Target Entity” : “XYZ,
        “Mapping” : [
        “Field 1” : {
          “Source Fields” : “PatientID”,
          “Target Field” : “Therefore, disclosed embodiments
may provide a cloud-based implementation thatPatient
Number/Custom Field”,
          “Transformations” : {
          “Name” : ,
          “Logic” :
          }
        }
        “Field 2” : {
          “Source Fields” : ,
          “Target Field” : ,
          “Transformations” : {
          “Name” : ,
          “Logic” :
          }
        }]
      }
        {
        “Source Entity” : “ABC”,
        “Target Entity” : “XYZ,
        “Mapping” : [{
          “Source Fields” : ,
          “Target Field” : ,
          “Transformations” : {
          “Name” : ,
          “Logic” :
          }
        }]
      }]
  }
  “Other Services” : {
    “ID” : “.....”,
    “Service Name” : “Pathways/.......”,
    “API”. “REST/SOAP/SOA/.......”,
    “URL”: “http://xyz.com”,
    “EndPoints” : {
        “Get” : “http://xyz.com”,
        “Post” : “http://xyz.com”
    }
    “Authentication” : “xyz”,
    “Data Load Mappings” : [
        {
        “Source Entity” : “ABC”,
        “Target Entity” : “XYZ,
        “Mapping” : [
        “Field 1” : {
          “Source Fields” : “....”,
          “Target Field” : “.....”,
          “Transformations” : {
          “Name” : ,
          “Logic” :
          }
        }
        “Field 2” : {
          “Source Fields” : ,
          “Target Field” : ,
          “Transformations” : {
          “Name” : ,
          “Logic” :
          }
        }]
      }
        {
        “Source Entity” : “ABC”,
        “Target Entity” : “XYZ,
        “Mapping” : [{
          “Source Fields” : ,
          “Target Field” : ,
          “Transformations” : {
          “Name” : ,
          “Logic” :
          }
        }]
      }]
  }
}
```

APPENDIX H

```
{
  “Entity 1” : {
    “Events” : [
    {
        “Event Name/Title” : “XYZ”,
        “Type” : “ABC”,
        “Description” : “....”,
        “System Action” : “.....”,
        “Workflow Actions: : {
        “Workflow Name” : “......”,
        “Subject” : “..........”,
        “Due Date” : “..........”;
        “Priority” : “...........”;
        “User Assignment” : “...........”,
        “Entity” : {
          “Id” : “..........”,
          “Type” : “........”,
          “Name” : “........”
        }
      }
    }
        {
        “Event Name/Title” : “XYZ”,
        “Type”: “ABC”,
        “Description” : “....”,
        “System Action” : “.....”,
        “Workflow Actions: : {
        “Workflow Name” : “......”,
        “Subject” : “..........”,
        “Due Date” : “..........”;
        “Priority” : “...........”;
        “User Assignment” : “...........”,
        “Entity” : {
          “Id” : “..........”,
          “Type” : “........”,
          “Name” : “........”
        }
      }
    }]
  }
  “Entity n” : {
    “Events”: [
    {
        “Event Name/Title” : “XYZ”,
        “Type” : “ABC”,
        “Description” : “....”,
        “System Action” : “.....”,
        “Workflow Actions: : {
        “Workflow Name” : “......”,
        “Subject” : “..........”,
        “Due Date” : “..........”;
        “Priority” : “...........”;
        “User Assignment” : “...........”,
        “Entity”: {
          “Id” : “..........”,
          “Type” : “........”,
          “Name” : “........”
        }
      }
    }
        {
        “Event Name/Title” : “XYZ”,
        “Type” : “ABC”,
        “Description” : “....”,
        “System Action” : “.....”,
        “Workflow Actions: : {
        “Workflow Name” : “......”,
        “Subject” : “..........”,
        “Due Date” : “..........”;
        “Priority” : “...........”;
        “User Assignment” : “...........”,
        “Entity” : {
          “Id” : “..........”,
          “Type” : “........”,
          “Name” : “........”
        }
      }
    }]
  }
}
```

APPENDIX I

```
{
  “Events”: {
  {
      “Event Name/Title” : “XYZ”,
      “Type” : “ABC”,
      “Description” : “....”,
      “System Action” : “.....”,
      “System Conditions” : {
        “Condition 1”: “.....”;
        “Condition n” : “.....”;
      }
      “Stage” : {
      “Parent Stage” : “Office Visit/Diagnosis”;
      “Child Stage” : “Null”,
      “Grand Child Stage” : “Null”,
      }
      “Target Actions: {
        “Id” : “..........”,
        “Type” : “........”,
        “Name” : “........”
        “Description” : “.......”
      }
    }
  {
      “Event Name/Title”: “XYZ”,
      “Type” : “ABC”,
      “Description” : “....”,
      “System Action” : “.....”,
      “System Conditions” : {
        “Condition 1”: “.....”;
        “Condition n”: “.....”;
      }
      “Stage” : {
      “Parent Stage” : “Office Visit/Diagnosis”;
      “Child Stage” : “Null”,
      “Grand Child Stage” : “Null”,
      }
      “Target Actions: {
        “Id” : “..........”,
        “Type” : “........”,
        “Name” : “........”
        “Description” : “.......”
      }
    }
```

What is claimed:

1. A system for performing a plurality of patient-related functionalities implemented in a cloud computing environment, the system comprising:
one or more software algorithms that facilitate at least one of patient onboarding, case management, task management and next best action evaluation of health benefits and prior authorization, treatment planning, and patient adherence metric analysis;
a controller implemented in a processor of a server, wherein the one or more software algorithms are run under the control of a controller implemented in the processor to analyze information utilized and/or generated by the one or more software algorithms; and
a memory for storing the information utilized by and/or generated by the one or more software algorithms to facilitate the patient-related functionalities.

2. The system of claim 1, wherein the patient-related functionalities are insured related functionalities.

3. The system of claim 1, wherein the patient-related functionalities include a patient onboarding process, wherein a referral processing utility leverages real time integration with a plurality of incoming referral methods.

4. The system of claim 1, wherein a plurality of patient view user interface screens are provided that enable input of data associated with a patient and review of such and related information accessed via that input.

5. The system of claim 4, wherein the plurality of patient view user interface screens include a case view dashboard configured to enable input of various data associated with a particular case and review of such and related information accessed via that input.

6. The system of claim 4, wherein an audit trail on the case view dashboard is configured to enable input of various data associated with a patient's case and review of such and related information accessed via that input.

7. The system of claim 4, wherein the case view dashboard includes health care professional or provider information on a particular case and review of such and related information accessed via that input.

8. The system of claim 1, wherein the patient-related functionalities are implemented with a front end that includes a user interface that includes a plurality of web and mobile device based screens providing input and/or output functionality to a user for inputting and/or reviewing output data regarding one or more of the patient-related functionalities.

9. The system of claim 8, wherein the user interface includes display and or receipt of data that enables provisioning of patient services in a cloud environment wherein an artificial intelligence driven hub that maps data from pharmaceutical and therapeutic prescribers, patients, care givers, payors, and pharmaceutical and therapeutic manufacturers, for patient care.

10. The system of claim 1, wherein the patient-related functionalities are implemented with a front end that includes a user interface that includes a plurality of web and mobile device based screens providing input and/or output functionality to a user for inputting and/or reviewing output data regarding one or more of the patient-related functionalities.

11. A method for performing a plurality of patient-related functionalities implemented in a cloud computing environment, the method comprising:
facilitating, using one or more software algorithms, at least one of patient onboarding, case management, task management and next best action evaluation of health benefits and prior authorization, treatment planning, and patient adherence metric analysis;
controlling the one or more software algorithms using a controller implemented in a processor of a server, wherein the controller is implemented in the processor to analyze information utilized and/or generated by the one or more software algorithms; and
storing, in a memory, the information utilized by and/or generated by the one or more software algorithms to facilitate the patient-related functionalities.

12. The method of claim 11, wherein the patient-related functionalities are insured related functionalities.

13. The method of claim 11, wherein the patient-related functionalities include a patient onboarding process, wherein a referral processing utility leverages real time integration with a plurality of incoming referral methods.

14. The method of claim 11, wherein a plurality of patient view user interface screens are provided that enable input of data associated with a patient and review of such and related information accessed via that input.

15. The method of claim 14, wherein the plurality of patient view user interface screens include a case view dashboard configured to enable input of various data associated with a particular case and review of such and related information accessed via that input.

16. The method of claim 14, wherein an audit trail on the case view dashboard is configured to enable input of various data associated with a patient's case and review of such and related information accessed via that input.

17. The method of claim 14, wherein the case view dashboard includes health care professional or provider information on a particular case and review of such and related information accessed via that input.

18. The method of claim 11, wherein the patient-related functionalities are implemented with a front end that includes a user interface that includes a plurality of web and mobile device based screens providing input and/or output functionality to a user for inputting and/or reviewing output data regarding one or more of the patient-related functionalities.

19. The method of claim 11, wherein the patient-related functionalities are implemented with a front end that includes a user interface that includes a plurality of web and mobile device based screens providing input and/or output functionality to a user for inputting and/or reviewing output data regarding one or more of the patient-related functionalities.

20. The method of claim 19, wherein the user interface includes display and or receipt of data that enables provisioning of patient services in a cloud environment wherein an artificial intelligence driven hub that maps data from pharmaceutical and therapeutic prescribers, patients, care givers, payors, and pharmaceutical and therapeutic manufacturers, for patient care.
